(19) United States
(12) Patent Application Publication
(10) Pub. No.: US 2012/0014912 A1
(43) Pub. Date: Jan. 19, 2012

(54) PALATABLE PHARMACEUTICAL COMPOSITION

(75) Inventor: Eleni Dokou, Cambridge, MA (US)

(73) Assignee: VERTEX PHARMACEUTICALS INCORPORATED, Cambridge, MA (US)

(21) Appl. No.: 13/182,695

(22) Filed: Jul. 14, 2011

Related U.S. Application Data
(60) Provisional application No. 61/364,090, filed on Jul. 14, 2010.

(51) Int. Cl. A61K 31/497; A61K 38/21; A61K 31/7056; A61P 31/14

(52) U.S. Cl. 424/85.4; 514/255.05; 514/43

(57) ABSTRACT

A pharmaceutical formulation comprising:
VX-950; and
a taste improving composition.

### CROSS-REFERENCE

The present application claims priority to U.S. Application No. 61/364,090 filed on Jul. 14, 2010, the contents of which are incorporated herein by reference in its entirety.

### FIELD OF THE INVENTION

The present invention relates to novel palatable pharmaceutical composition of VX-950 for oral administration. This invention also relates to palatable pharmaceutical compositions comprising VX-950, a taste improving composition and one or more excipients.

### BACKGROUND OF THE INVENTION

Hepatitis C virus (HCV) is estimated to infect 170 million people worldwide [Purcell, R. H., Hepatitis C virus: historical perspective and current concepts. FEMS Microbiology Reviews, 1994. 14: p. 181-192.] Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31, (Suppl. 1), pp. 88-91 (1999)].

VX-950 is a competitive, reversible peptidomimetic hepatitis C virus (“HCV”) NS3/4A protease inhibitor with a steady state binding constant (ki*) of 3 nM (and with a Ki of 8 nM) [See International Publication No. 02/018369].

In clinical trials, VX-950 has shown antiviral activity been shown to be an effective therapy against HCV, which is recognized as the causative agent for most cases of non-A, non-B hepatitis, with an estimated human sero-prevalence of 3% globally [A. Alberti et al., “Natural History of Hepatitis C,” J. Hepatology, 31., (Suppl. 1), pp. 17-24 (1999)].

Orally administerable a drug including VX-950 can be provided to the patient in various dosage forms, including formations such as capsules, caplets, tablets and other solid forms. Swallowing such solid forms is a problem for many people including children and geriatric patients. For example, when the solid form of the drug is large, swallowing such drug form can be difficult. To facilitate a less burdensome administration of such solid forms of drugs for affected patients, chewable formulations have been conceived. For a chewable formulation, palatability (e.g., aroma, taste, texture and mouthfeel) is extremely important in attaining acceptable dosing compliance, particularly in pediatric and geriatric patients. The palatability is further magnified in therapies that require multiple (twice or three-times daily) dosing over a multi-month therapy period. Additional problems arise when the chewable formulations contain a bitter tasting active pharmaceutical ingredient. For example, VX-950 and excipients necessary to formulate VX-950 were found to be very bitter with a bitterness that lingers for an extended period of time with the aftertaste. In addition to bitter taste, VX-950 leaves a dry mouthfeel and/or mouth irritation when they are orally digested. Various materials have been incorporated in chewable formulations to diminish the bitter taste of VX-950.

As such, there is a continued need to find palatable formulations that diminish the bitter taste of VX-950 and/or of accompanying excipients be administered with ease and that affected patients are more inclined to comply with their medication instructions.

### SUMMARY OF THE INVENTION

The present invention provides a formulation for diminishing bitter taste of VX-950.

In one aspect, the present invention is a pharmaceutical formulation comprising VX-950 and a taste improving composition.

In one embodiment, the wt. % ratio of VX-950 with respect to the taste improving composition ranges from about 20:1 to about 1:2. In some embodiments, the wt. % ratio of VX-950 with respect to the taste improving composition ranges from about 15:1 to about 10:1.

In one embodiment, VX-950 is in an amorphous form. In some embodiments, VX-950 is spray-dried with a polymer. in some embodiments, the spray-dried dispersion contains 49.5% of VX-950, 49.5% of hydroxypropylmethylcellulose acetate succinate (HPMCAS) and 1% sodium lauryl sulfate (SLS).

In one embodiment, the taste improving composition includes one or more of a flavoring agent and a sweetener. In one embodiment, the flavoring agent is a natural flavor, an artificial flavor, or both. In some embodiments, one or more flavoring agents are selected from the group consisting of: spearmint oil, cinnamon oil, oil of wintergreen, peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, natural and artificial orange flavor and a fruit flavor and a combination thereof. In some embodiments, the flavor agent is ethyl vanillin, a fruit flavor or both.

In one embodiment, one or more fruit flavors are selected from the group consisting of: natural and/or artificial flavor of apple, pear, peach, orange, grape, strawberry, raspberry, cherry, plum, pineapple, and apricot. In some embodiments, the fruit flavor is natural and artificial orange.

In one embodiment, the pharmaceutical formulation comprises from about 0 wt. % to about 5 wt. % of the one or more flavoring agents. In some embodiments, the pharmaceutical formulation comprises from about 1 wt. % to about 3 wt. % of the one or more flavoring agents.

In one embodiment, one or more sweeteners are selected from the group consisting of: xylose, glucose, sucralose, mannose, spartane, neotame, sucralose, alitame, dextrose, fructose, maltitol, lactitol, xylitol, trehalose, tagatose, erythritol, isomalt, maltose, neohesperidin dihydrochalcone, sodium cyclamate, thaumatin, sodium saccharin, saccharin, galactose, fructose, dextrose, lactose, trehalose, lactosucrose, erythritol, sucrose, maltose, sorbitol, xylitol, mannitol, glycerin, aspartame, acesulfame potassium, cyclamate, saccharin, and saccharin sodium. In some embodiments, the sweetener is sucralose, aspartame or both. In some embodiments, the sweetener is sucralose.

In one embodiment, the pharmaceutical formulation comprises from about 1 wt. % to about 4 wt. % of the one or more sweeteners. In some embodiments, the pharmaceutical formulation comprises from about 1 wt. % to about 2 wt. % of the one or more sweeteners.

In one embodiment, the pharmaceutical formulation comprises from about 20 wt. % to about 80 wt. % of VX-950. In some embodiments, the pharmaceutical formulation comprises from about 40 wt. % to about 60 wt. % of VX-950. In some embodiments, the pharmaceutical formulation comprises about 50 wt. % of VX-950. In another embodiment, VX-950 is in the form of a spray-dried dispersion. In yet another embodiment, the pharmaceutical formulation is a tablet (e.g., a 250 mg or 100 mg tablet) including spray-dried VX-950. In some embodiments, the 100 mg tablet is the same blend formulation as the 250 mg tablet, for example as shown in Tables 1 and 5.

In one embodiment, the pharmaceutical formulation further includes one or more excipients. In some embodiments, the one or more excipients is a filler, a glidant, a lubricant, a disintegrant, or a combination thereof.

In one embodiment, the disintergrant includes one or more ingredients selected from the group consisting of croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate. In some embodiments, the disintegrant is croscarmellose sodium.

In one embodiment, the pharmaceutical formulation comprises from about 1 wt. % to about 5 wt. % of the disintegrant. In some embodiments, the pharmaceutical formulation comprises about 3 wt. % of the disintegrant.

In one embodiment, the lubricant includes one or more ingredients selected from the group consisting of: talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, and sodium benzoate. In some embodiments, the lubricant is sodium stearyl fumarate.

In one embodiment, the pharmaceutical formulation comprises from about 1 wt. % to about 5 wt. % of the lubricant. In some embodiments, the pharmaceutical formulation comprises about 3 wt. % of the lubricant.

In one embodiment, the filler includes one or more ingredients selected from the group consisting of lactose, dextrose, maltodextrin, sorbitol, xylitol, mannitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate. In some embodiments, the filler is mannitol, microcrystalline cellulose or both.

In one embodiment, the filler comprises from about 20 wt. % to about 50 wt. %. In some embodiments, the filler comprises from about 35 wt. % to about 45 wt. %. In some embodiments, the filler comprises from about 38 wt. % to about 41 wt. %.

In one embodiment, the one or more excipients further includes a colorant. In some embodiments, the colorant includes one or more ingredients selected from the group consisting of red, black and yellow iron oxides, and FD & C dyes.

In one embodiment, the one or more excipients further includes a glidant. Examples of the glidants may include, but are not limited to, talc, colloidal silica (e.g., Cabosil M-5), magnesium oxide, magnesium silicate, leucine and starch. In one embodiment, the one or more glidants is colloidal silica.

In one embodiment, the one or more excipients is one or more selected from the group consisting of croscarmellose sodium, sodium stearyl fumarate, microcrystalline cellulose, red and yellow iron oxides, mannitol and silicon dioxide (colloidal silica).

In one embodiment, the pharmaceutical formulation is in a form of a capsule, tablet, pill, powder, granule, or aqueous suspension or solution. In some embodiments, the formulation is in a form of a tablet. In some embodiments, the tablet is chewable.

In one embodiment, the intensity of the bitterness of the pharmaceutical formulation, when administered, is at least 30% less than a VX-950 formulation without the taste improving composition. Example 4 shows the bitterness profile of a palatable formulation of the present invention. In some embodiments, the intensity of the bitterness of the pharmaceutical formulation is at least 50%, 10 min after administration, less than a VX-950 formulation without the taste improving composition. In some embodiments, the intensity of the chalky/dry mouthfeel of the pharmaceutical formulation is at least 50% less than a VX-950 formulation without the taste improving composition, 10 min after administration.

In one aspect of the present invention, the present invention is a method of preparing a pharmaceutical formulation comprising:
a) blending VX-950 with a taste improving composition and one or more excipients;
b) forming a blended mixture; and
c) lubricating the blended mixture.

In one embodiment of the method, the intensity of the bitterness of the pharmaceutical formulation, when administered, is at least 30% less than a VX-950 formulation without the taste improving composition.

In one embodiment of the method, the intensity of the chalky/dry mouthfeel of the pharmaceutical formulation is at least 50% less than a VX-950 formulation without the taste improving composition, 10 min after administration.

In one embodiment of the method, the intensity of the chalky/dry mouthfeel of the pharmaceutical formulation is at least 50% less than a VX-950 formulation without the taste improving composition, 10 min after administration.

In one aspect of the present invention, the pharmaceutical formulation comprises:
a) a means for blending VX-950 with a taste improving composition and one or more excipients;
b) a means for forming a blended mixture; and
c) a means for lubricating the blended mixture.

In one embodiment, the intensity of the bitterness of the pharmaceutical formulation, when administered, is at least 30% less than a VX-950 formulation without the taste improving composition.

In one embodiment, the intensity of the chalky/dry mouthfeel of the pharmaceutical formulation is at least 50% less than a VX-950 formulation without the taste improving composition, 10 min after administration.

In one embodiment, the invention provides a pharmaceutical formulation comprising: a) VX-950 in a spray-dried dispersion; b) ethyl vanillin; c) natural and artificial orange flavor; and d) sucralose.

In one embodiment, the invention provides a method of administering a palatable pharmaceutical formulation of VX-950 to a patient infected with hepatitis C. In one embodiment, the dosage of a pharmaceutical formulation of the present invention per administration is in an amount of about 250 mg to about 2250 mg VX-950. In one embodiment, the dosage of a pharmaceutical formulation of the present invention per administration is in an amount of about 300 mg to about 1500 mg VX-950. In one embodiment, the dosage of a pharmaceutical formulation of the present invention per administration is in an amount of about 300 mg to about 1250 mg VX-950. In some embodiments, the pharmaceutical formulation per administration is: a) in an amount of 250 mg VX-950; b) in an amount of 300 mg VX-950; c) in an amount of 400 mg VX-950; d) in an amount of 450 mg VX-950; e) in an amount of 500 mg VX-950; f) in an amount of 600 mg VX-950; g) in an amount of 650 mg VX-950; h) in an amount of 750 mg VX-950; i) in an amount of 850 mg VX-950; j) in an amount of 1000 mg VX-950; k) in an amount of 1250 mg VX-950; or in an amount of 2250 mg VX-950. In some embodiments, the pharmaceutical formulation is administered once per day, twice per day or three times per day.

In some embodiments, the pharmaceutical formulation is administered in an amount of 10-20 mg VX-950 per administration per kg of body weight. In some embodiments, the pharmaceutical formulation is administered in an amount of 15-18 mg VX-950 per administration per kg of body weight. In some embodiments, the pharmaceutical formulation is administered: a) in an amount of 15 mg VX-950 per kg of body weight; b) in an amount of 16 mg VX-950 per kg of body weight; or c) in an amount of 18 mg VX-950 per kg of body weight.

In some embodiments, the invention further provides administering one or more additional antiviral agents. In some embodiments, the one or more antiviral agents are pegylated-interferon and ribavirin.

All of the documents cited herein, are incorporated herein by reference in their entireties.

### DESCRIPTION OF THE FIGURES

FIG. 1 shows the manufacturing process of VX-950 orange chewable tablet product.

FIG. 2 shows the effect of VX-950 chewable tablet hardness on dissolution in 1% SLS.

FIG. 3 shows the dissolution profile of the 250 mg VX-950 chewable tablet in 1% SLS.

FIG. 4 shows the bitterness profile results of:

(a) the VX-950 tablet of Table 2 (“Adult Tablet”);

(b) a VX-950 blended with sucralose & ethyl vanillin in Table 3; and

(c) a taste improving VX-950 formulation of Table 1.

FIG. 5 shows the chalky/dry mouthfeel profile results of:

(a) the VX-950 tablet of Table 2 (“Adult Tablet”);

(b) a VX-950 blended with sucralose & ethyl vanillin in Table 3; and

(c) a taste improving VX-950 formulation of Table 1.

### DESCRIPTION OF THE INVENTION

The pharmaceutical formulations of the present invention can be used as a delivery system for the administration of one or more APIs. Any suitable API can be used in accordance with the present invention.

VX-950 is described in PCT Publication Numbers WO 02/018369, WO 2006/050250 and WO/2008/144072, with reference to the following structural formula, or a pharmaceutically acceptable salt thereof:

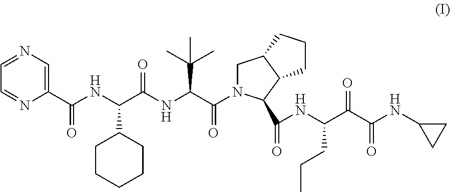

Other descriptions of VX-950 can be found in PCT Publication Numbers WO 07/098,270 and WO 08/106,151.

As used herein, the term “VX-950,” refers to the compound of Formula (I), or a pharmaceutically acceptable salt thereof. Further, the term “VX-950” can also include a processed form of VX-950. For example, a VX-950 spray-dried dispersion that includes VX-950 and a polymer(s) is encompassed within the term. A spray-dried dispersion of VX-950 is described in WO 05/123076, WO 07/109,604, WO 07/109,605 and WO 08/080,167.

In one aspect of the invention, VX-950 is in the form of a spray dried dispersion. The term “spray dried” or “spray drying” in the present specification means the state of the drug alone or the drug together with a pharmaceutically acceptable carrier dissolved in a solvent that is pharmaceutically acceptable, or suspended with the drug or part or all of the carrier dispersed in a solvent and this solution or suspension being sprayed and dried.

Spray drying of the pharmaceutical compositions may be undertaken utilizing either rotary, pneumatic or pressure atomisers located in either a co-current, counter-current or mixed-flow spray dryer or variations thereof.

In one aspect of the present invention, the spray dried dispersed VX-950 is mixed with a taste improving composition and one or more excipients, forming a pharmaceutical formulation of the present invention.

The amount of VX-950 in the formulation of the present invention can be expressed in terms of a weight percentage. For example, the active ingredient in the formulation of the present invention can constitute from greater than 0% to about 80% by weight based on the total weight of the formulation, or from greater than 0% to about 60% by weight based on the total weight of the formulation. The amount of VX-950 in the formulation of the present invention also can be expressed in terms of total mass of the formulation. For example, the formulation of the present invention can include VX-950 in an amount of from about 1 μg to about 2 g per tablet, or from about 0.01 mg and about 1000 mg per tablet. In another example, the formulation of the present invention can include one or more active ingredients in amounts of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. In some embodiments, the formulation of the present invention can include one or more active ingredients in amounts of about 100 mg, about 250 mg. Or for example, the formulation of the present invention can include one or more active ingredients in amounts that range, e.g., from about 0.1 mg to about 0.5 mg, from about 1 mg to about 20 mg (e.g., 2 mg, 8 mg, 15 mg), from about 50 mg to about 100 mg (e.g., 80 mg), from about 100 mg to about 500 mg (e.g., 100 mg, 200 mg, 250 mg, 300 mg), from about 100 to 200 mg, from about 100 mg to 150 mg, from about 100 mg to about 125 mg, from about 200 mg to about 300 mg, from 200 mg to about 250 mg, from 225 mg to 250 mg, from about 225 mg to about 250 mg, from about 240 mg to about 250 mg, or from about 500 mg to about 1000 mg.

In some embodiments of the present invention, the taste improving formulation of VX-950 is a pediatric formulation. The dosage and frequency of administration will depend on the age, sex and condition of the pediatric patient, concurrent administration of other drugs, counter indications and other parameters to be taken into account by the clinician.

The pharmaceutical formulations of this invention may be orally administered in any orally acceptable dosage form including, but not limited to, capsules, sprinkles, tablets, aqueous suspensions or solutions. In one embodiment of the present invention, the pharmaceutical formulation is in form of a tablet. Furthermore, in one embodiment, a tablet form can be a chewable, orally disintegrating and/or rapidly disintegrating form.

The term “tablet” refers to a pharmacological composition in the form of a small, essentially solid pellet of any shape. Tablet shapes can be cylindrical, spherical, rectangular, capsular or irregular. The term “tablet composition” refers to the substances included in a tablet. A “tablet composition constituent” or “tablet constituent” refers to a compound or substance which is included in a tablet composition. These can include, but are not limited to, the active ingredient and one or more excipients in addition.

In some embodiment, the tablet is chewable.

The amounts of VX-950 according to this invention are administered in a single dosage form or in more than one dosage form. If in separate dosage forms, each dosage form is administered about simultaneously. For the avoidance of doubt, for dosing regimens calling for dosing more than once a day, one or more tablet or dose may be given at each time per day (e.g., 1 tablet, twice per day, 2 tablets, twice per day or 3 tablets, twice per day).

Methods of forming the tablets of the invention wherein all tablet constituents are combined simultaneously or wherein any combination of tablet constituents are combined separate from the other constituents are within the scope of the invention.

VX-950 and excipient(s) mixture can be prepared by, for instance, conventional mixing, compacting, granulating, compression, or coating. Procedures which may be used are known in the art, e.g., those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed. (Mack Publ., Co., 1970) or later editions. Examples of such techniques are as follows:

(1) Blending of VX-950 with the appropriate excipients using different blending equipment, such low shear blenders and high shear blenders;

(2) Direct compression of the blends, using appropriate punches and dies; the punches and dies are fitted to a suitable compaction machine, such as rotary tableting press or a single station compaction machine;

(3) The formulation blend can be granulated if necessary, using appropriate granulation methods such as dry granulation (slugging or roller compaction), high shear wet granulation, fluid bed granulation, extrusion-spheronization etc;

(4) Granulation followed by compression; and

(5) Coating of the tablets, if necessary, produced using appropriate coating equipment (e.g., coating pans) and appropriate coating solutions/suspensions to be applied on the tablets.

In one aspect of the present invention, the formulations of the present invention find their greatest utility when administered to a subject who is in the fed or fasted state, preferably in the fed state.

The tablets may be produced by way of a conventional method or combinations of conventional methods such as roller compaction and compression method. For example, a tableting process is essential for production methods of tablets, and also the other processes such as of mixing, drying, and coating may be combined as required. The tableting process may be, for example, a direct compression method where VX-950 and pharmaceutically acceptable excipients disclosed herein are mixed and then the mixture is compressed into tablets by use of tableting machines.

In one embodiment of the invention, the tablet has a hardness in the range of about 4 to 20 kp (kilopond). The tablet of this embodiment may or may not comprise an outer coating as described below.

Once tablet compositions are prepared, they may be formed into various shapes. In some embodiments, the tablet compositions are pressed into a shape. This process may comprise placing the tablet composition into a form and applying pressure to the composition so as to cause the composition to assume the shape of the surface of the form with which the composition is in contact. In some embodiments, the tablet has a hardness in the range of about 10 to 20 kp. In some embodiments, the tablet has a hardness in the range of about 8 to 13 kp.

Yet in one embodiment of the present invention, the formulation includes tablet compositions that may be coated.

The present invention can also provide a formulation that diminishes the bitter taste, mouth irritation, and dry mouthfeel when a patient is administered with VX-950.

The present invention is suitable for rendering VX-950 that are bitter tasting and/or throat catching. Taste improving compositions would diminish any off-flavors in the taste of VX-950, and to also improve the taste of any other off-flavor components included in the formulation if desired.

The term “taste improving” referred herein can be defined as a perceived reduction of an undesirable taste that would otherwise be there to making it possible to delay or diminish the occurrence of an unpleasant taste specific to a product during its oral, buccal or nasal administration.

The present invention is not limited to the recited amount but rather a taste improving effective amount, whereby the taste of VX-950, which is bitter tasting, is masked and the pharmaceutical formulation is taste improving to the intended patient, such as a pediatric or geriatric patient in need thereof.

In one embodiment, the taste improving composition can include one or more components. In some embodiments, the taste improving composition can include one or more sweeteners. In some embodiments, the taste improving composition can include one or more flavoring agents. In some embodiments, the taste improving composition can include a combination of a sweetener and a flavoring agent.

Taste improving composition can be used in conventional amounts and in one embodiment, in an amount of about 0 to about 99% by total weight of the formulation and in one embodiment, in an amount of about 1% to about 50% by weight of the formulation, and in some embodiments, in an amount of about 2% to about 50% by weight of the formulation. In some embodiments, the formulation can include about 30% to about 50% of the taste improving composition.

In some embodiments, one or more sweeteners include, but are not limited to, monosaccharides, disaccharides and polysaccharides. Examples of suitable sweeteners include both natural and artificial sweeteners. Examples can include, but are not limited to, glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartane, neotame, alitame, neohesperidin dihydrochalcone, sodium cyclamate, thaumatin, acesulfame potassium, saccharin, and saccharin sodium.

In one embodiment, a sweetener is aspartame, sucralose, or a combination thereof. In some embodiments, the sweetener is sucralose. In some embodiments, the composition of the present invention can include a combination of aspartame and sucralose. The amount of sweetener used in the taste improving composition will vary depending on the degree of palatability desired for the pharmaceutical formulation. In one embodiment, the amount of a sweetener used in the taste improving composition has a range of from about 0 wt. % to about 5 wt. %. In some embodiments, the amount of a sweetener used in the taste improving composition has a range of from about 0 wt. % to about 2 wt. %. In some embodiments, the amount of a sweetener used in the taste improving composition has a range of from about 1 wt. % to about 3 wt. %.

The flavoring agent used is of the type and amount desired to enhance the palatability of the particular liquid pharmaceutical composition to the intended consumer. The flavoring agent used for a solid formulation is similar.

Suitable flavoring agents can include, for example, flavors, which are known to those of skill in the art, such as, for example, natural flavors, artificial flavors, and combinations thereof. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavoring agents may be used in liquid or solid form and, as indicated above, may be used individually or in admixture. Other flavoring agents can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof. In one embodiment, the flavoring agent of the present invention is ethyl vanillin, natural & artificial orange flavor or both. A formulation of the present invention can include from about 0 wt. % to about 5 wt. % of the flavor agent. In some embodiments, a formulation of the present invention can include from about 1 wt. % to about 3 wt. % of the flavor agent. In some embodiments, a formulation of the present invention can include from about 2 wt. % to about 3 wt. % of the flavor agent.

The term “filler component” refers to one or more substances that act to dilute the API to the desired dosage and/or that act as a carrier for the API. An “excipient” can also refer to a non-toxic pharmaceutically acceptable substance added to a pharmacological composition to facilitate the processing, administration and pharmaceutics properties of a compound. Excipients that are pharmaceutically acceptable and are used as additives can also be added to the pharmaceutical formulations of the present invention. Examples of these excipients are a filler/diluent (extender)/binder, disintegrant, sweetener, flavoring agent, lubricant, glidant, surfactant, coloration agent or a combination thereof. One or a combination of 2 or more of these excipients can be used. Other excipients include e.g. coloring agents, pH-adjusting agents, buffering agents, preservatives, anti-oxidants, wetting agents, humidity-adjusting agents, surface-active agents, suspending agents, absorption enhancing agents, foaming agents, agents for modified release and mixtures thereof. Generally, excipients forth may be used for customary purposes and in typical amounts without adversely affecting the properties of the compositions. These excipients may be utilized in order to formulate the composition into tablets, capsules, and other solid forms. In some embodiments, the filler component comprises at least one of a substance that improves the mechanical strength and/or compressibility of the pharmaceutical compositions of the invention.

Examples of the filler can include, but are not limited to, mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate. In one embodiment, the filler is mannitol, microcrystalline cellulose, or a combination thereof.

In some embodiments, the filler is present in an amount of about at least 1, 2, 3, 4, 5, 6, 7, 8, 9, 10, 11, 12, 13, 14, 15, 20, 25, 30, 35, 40, 45 or 50% of the total weight of the formulation.

In certain embodiments, the pharmaceutical compositions of the present invention comprise a first filler and a second filler. In some embodiments of the pharmaceutical formulations, each of the first and second filler component independently comprises from about 0.01% to about 30% by weight of the pharmaceutical formulation. In some embodiments of the pharmaceutical formulations, each of the first and second filler component independently comprises from 5% to about 25% by weight of the pharmaceutical formulation. In some embodiments of the pharmaceutical formulations, each of the first and second filler component independently comprises from about 10% to about 20% by weight of the pharmaceutical formulation. In some embodiments of the pharmaceutical formulations, each of the first and second filler component independently comprises from about 15% to about 20% by weight of the pharmaceutical formulation.

“Disintegrants” are substances that are added to a tablet to facilitate its breakup or disintegration after administration. Examples of the disintegrants may include, but are not limited to, croscarmellose sodium (e.g., AcDiSol), sodium alginate, calcium alginate, alginic acid, starch, pregelatinized starch, sodium starch glycolate, crospovidone, carboxymethylcellulose calcium, cellulose and its derivatives, carboxymethylcellulose sodium, soy polysaccharide, guar gum, an ion exchange resin, an effervescent system based on food acids and an alkaline carbonate component, and sodium bicarbonate. In some embodiments of the pharmaceutical formulations, the disintegrant component is croscarmellose sodium.

In some embodiments of the pharmaceutical formulations, the disintegrant component comprises an amount of about at least 1, 2, 3, 4, 5, 6, 7, 8, 9, 10, 15, 20, 25, 30, 35 or 40% of the total weight of the formulation. In some embodiments of the pharmaceutical formulations, the disintegrant component comprises from about 0.01% to about 30% by weight of the pharmaceutical formulation. In some embodiments, the disintegrant component comprises from about 0.01% to about 20% by weight of the pharmaceutical formulation. In some embodiments, the disintegrant component comprises from about 0.5% to about 20% by weight of the pharmaceutical formulation. In some embodiments, the disintegrant component comprises from about 0.1% to about 20% by weight of the pharmaceutical formulation. In some embodiments, the disintegrant component comprises from about 0.5% to about 15% by weight of the pharmaceutical formulation. In some embodiments, the disintegrant component comprises from 0.5% to about 10% by weight of the pharmaceutical formulation. In some embodiments, the disintegrant component comprises from about 0.5% to about 5% by weight of the pharmaceutical formulation. In some embodiments, the disintegrant component comprises from about 1% to about 4% by weight of the pharmaceutical formulation. In some embodiments, the disintegrant component comprises from about 1% to about 3% by weight of the pharmaceutical formulation. In some embodiments, the disintegrant component comprises from about 2% to about 3% by weight of the pharmaceutical formulation. In some embodiments, the disintegrant component comprises about 3% by weight of the pharmaceutical formulation. In some embodiments, the disintegrant component comprises about 3% by weight of the pharmaceutical formulation.

A “glidant” is a substance to promote powder flow by reducing interparticle friction and cohesion. In certain embodiments, the one or more excipients can include one or more glidants. Examples of the glidants may include, but are not limited to, talc, colloidal silica (e.g., Cabosil M-5), magnesium oxide, magnesium silicate, leucine and starch. In one embodiment, the one or more glidants is colloidal silica. In one embodiment, the one or more glidants comprises about up to 3% by weight of the pharmaceutical formulation. In another embodiment, the one or more glidants comprises about up to 1% by weight of the pharmaceutical formulation. In another embodiment, the one or more glidants comprises about up to 0.5% by weight of the pharmaceutical formulation.

In certain embodiments, the one or more excipients can include one or more lubricants. Suitable lubricants possess anti-sticking or anti-tacking properties. Examples of the lubricants may include, but are not limited to, talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, or a combination thereof. In certain embodiment of the pharmaceutical formulations, the one or more lubricant is sodium stearyl fumarate.

In some embodiments of the pharmaceutical formulations, the one or more lubricant comprises an amount of about at least 1, 2, 3, 4, 5, 6, 7, 8, 9, 10, 15, 20, 25, 30, 35 or 40% of the total weight of the formulation. In some embodiment, the one or more lubricant comprises from about 0.01% to about 30% by weight of the pharmaceutical formulation. In some embodiments, the one or more lubricant comprises from about 0.01% to about 20% by weight of the pharmaceutical formulation. In some embodiments, the one or more lubricant comprises from about 0.1% to about 20% by weight of the pharmaceutical formulation. In some embodiments, the one or more lubricant comprises from about 0.5% to about 5% by weight of the pharmaceutical formulation. In some embodiments, the one or more lubricant comprises from about 1% to about 5% by weight of the pharmaceutical formulation. In some embodiments, the one or more lubricant comprises from about 0.5% to about 4% by weight of the pharmaceutical formulation. In some embodiments, the one or more lubricant comprises from about 1% to about 3% by weight of the pharmaceutical formulation. In some embodiments, the one or more lubricant comprises about 3% by weight of the pharmaceutical formulation.

Examples of colorants are food coloring, such as yellow food dye No. 5, red food dye No. 2, blue food dye No. 2, etc.; food lake coloring; iron oxides (e.g. iron oxide red), etc.

In some embodiments, the one or more colorant comprises an amount of about at least 0.1, 0.5, 1, 2, 3, 4, 5, 6, 7, 8, 9, 10, 15 or 20% of the total weight of the composition. In one embodiment, the one or more colorants comprises from about 1% to about 5% by weight of the pharmaceutical formulation. In some embodiments, the one or more colorants comprises from about 0.5% to about 4% by weight of the pharmaceutical formulation. In some embodiments, the one or more colorants comprises from about 1% to about 3% by weight of the pharmaceutical formulation. In some embodiments, the one or more colorants comprises from about 0.1% by weight to about 3.0% by weight of the pharmaceutical formulation. In one embodiment, the formulation of the present invention includes red and yellow iron oxides comprising about 0.5% of the total weight of the composition.

Furthermore, an excipient disclosed herein can have more than one function. For example, mannitol can function as a sweetener as a component of the taste improving composition and/or as a filler.

Each dosage form may be individually housed, as in a sheet of a metal foil-plastic laminate with each dosage form isolated from the others in individual cells or bubbles, or the dosage forms may be housed in a single container, as in a plastic bottle. In some embodiments, the VX-950 is packaged in foil pouches with a polypropylene heat seal layer. In some embodiments, the VX-950 is packaged in high density polyethylene (HDPE) bottles.

When compared to a formulation without a taste improving system described herein, a formulation of the present invention is found to be less bitter initially and in the aftertaste. The formulation also produced less mouth irritation, particularly in the aftertaste, and less chalky/drying mouthfeel.

In certain embodiments, a method according to this invention involves the treatment of a patient infected with genotype 1 Hepatitis C virus. In some embodiments, the patient is less than 18 years of age. In some embodiments, the patient is from 3 to 17 years of age. In some embodiments, the patient is from 18 to 50 years of age. In some embodiments, the patient is over 50 years of age.

In some embodiments, the patient is a treatment naïve patient. In other embodiments, the patient is a pegylated-interferon/ribavirin non-responder. As used herein “treatment naïve” refers to a patient who has not received any prior treatment for hepatitis C. As used herein “P/R non-responsive” includes patients who do not achieve or maintain a sustained virologic response (SVR) (undetectable HCV RNA 24 weeks after the completion of treatment) to the standard peg-IFN with RBV treatment, and patients who have had a lack of response. Lack of response is defined as a <2-log 10 decline from baseline in HCV RNA, as a failure to achieve undetectable levels of HCV virus, or as a relapse following discontinuation of treatment. As defined above, undetectable HCV RNA means that the HCV RNA is present in less than 10 IU/mL as determined by assays currently commercially available, for example, as determined by the Roche COBAS TaqMan™ HCV/HPS assay.

Any suitable dosage level of VX-950 can be employed in the formulations of the present invention. The dose to be administered to an animal, particularly a human, in accordance with the present invention should be sufficient to affect a therapeutic response in the animal over a reasonable time frame. One skilled in the art will recognize that the amount of active ingredient will vary depending upon a variety of factors including, for example, the activity of the specific compound employed; the age, body weight, general health, sex, and diet of a particular patient or patient population; the time of administration, rate of absorption, and rate of excretion; the potential interactions with other drugs taken separately or in combination; and the severity of the particular disease or condition for which a therapeutic effect is desired. The size of the dose will also be determined by the existence, nature, and extent of any adverse side effects that might accompany the administration of a particular compound. Other factors, which affect the specific dosage, include, for example, bioavailability, metabolic profile, and the pharmacodynamics associated with the particular compound to be administered in a particular patient.

For example, a pharmaceutically effective amount can include the amount or quantity of VX-950, which is sufficient to elicit the required or desired therapeutic response, e.g., an amount, which is sufficient to elicit a biological or therapeutic response when administered to a patient.

In some embodiments of this invention, VX-950, or a pharmaceutically acceptable salt thereof, alone or in a spray-dried dispersion, per administration is in an amount of about 250 mg to about 2250 mg. In some embodiments of this invention, VX-950, or a pharmaceutically acceptable salt thereof, per administration is in an amount of about 300 mg to about 1500 mg. In some embodiments of this invention, VX-950, or a pharmaceutically acceptable salt thereof, per administration is in an amount of about 250 mg to about 1250 mg.

In certain embodiments, the dose of VX-950 per administration is at least about 250 mg. In certain embodiments, the dose of VX-950 per administration is at least about 300 mg. In other embodiments, the dose of VX-950 per administration is at least about 400 mg. In other embodiments, the dose of VX-950 per administration is at least about 450 mg. In other embodiments, the dose of VX-950 per administration is at least about 500 mg. In other embodiments, the dose of VX-950 per administration is at least about 600 mg. In other embodiments, the dose of VX-950 per administration is at least about 650 mg. In other embodiments, the dose of VX-950 per administration is at least about 750 mg. In other embodiments, the dose of VX-950 per administration is at least about 850 mg. In other embodiments, the dose of VX-950 per administration is at least about 1000 mg. In other embodiments, the dose of VX-950 per administration is at least about 1125 mg. In other embodiments, the dose of VX-950 per administration is at least about 1250 mg. In other embodiments, the dose of VX-950 per administration is at least about 1500 mg.

In yet other embodiments, the dose of VX-950 per administration is no more than about 1500 mg. In other embodiments, the dose of VX-950 per administration is no more than about 1250 mg. In other embodiments, the dose of VX-950 per administration is no more than about 1125 mg. In other embodiments, the dose of VX-950 per administration is no more than about 1000 mg. In other embodiments, the dose of VX-950 per administration is no more than about 850 mg. In other embodiments, the dose of VX-950 per administration is no more than about 750 mg. In other embodiments, the dose of VX-950 per administration is no more than about 650 mg. In other embodiments, the dose of VX-950 per administration is no more than about 600 mg. In other embodiments, the dose of VX-950 per administration is no more than about 500 mg. In other embodiments, the dose of VX-950 per administration is no more than about 450 mg. In other embodiments, the dose of VX-950 per administration is no more than about 400 mg. In other embodiments, the dose of VX-950 per administration is no more than about 300 mg. In other embodiments, the dose of VX-950 per administration is no more than about 250 mg.

It should be understood that these lower and upper amounts may be combined to provide preferred dose ranges for administering VX-950. For example, in one embodiment, the VX-950, or the pharmaceutically acceptable salt thereof, per administration is in an amount of about 250 mg to about 2250 mg.

In some embodiments, the pharmaceutical formulation is administered in an amount of 10-20 mg VX-950 per administration per kg of body weight. In some embodiments, the pharmaceutical formulation is administered in an amount of 15-18 mg VX-950 per administration per kg of body weight. In some embodiments, the pharmaceutical formulation is administered: a) in an amount of 15 mg VX-950 per kg of body weight; b) in an amount of 16 mg VX-950 per kg of body weight; or c) in an amount of 18 mg VX-950 per kg of body weight.

In any of these embodiments, the amount of VX-950 is administered once a day. Alternatively, the amount of VX-950 is administered twice a day (e.g., BID; every 12 hours (q12h)). Alternatively, the amount of VX-950 is administered three-times per day (e.g., TID; every 8 hours (q8h)). VX-950 may be administered with or without food.

As would be recognized, it advantageous to have flexible dosing schedules. Accordingly, in some embodiments of this invention, the administration is 3 times per day, but not every 8 hours, or twice per day, but not every 12 hours.

In some embodiments, VX-950 is administered to a patient infected with HCV, such that the level of viral RNA in the patient is decreased to undetectable levels and remains at undetectable levels until a “sustained viral response” is achieved. As used herein, “sustained viral response” or “SVR” means that after dosing is completed, viral RNA levels remain undetectable.

This invention also provides a method for providing VX-950 to a human in need thereof, comprising administration to the human an oral dose of a composition comprising VX-950, wherein said dose provides to said human an average plasma concentration (Cavg) of VX-950 of at least about 250 ng/mL after the administration. In certain embodiments, the (Cavg) is about 1000 ng/mL, about 250 ng/ml, about 300 ng/ml, about 400 ng/ml, about 450 ng/ml, about 500 ng/ml, about 600 ng/ml, about 650 ng/ml, about 750 ng/ml, about 850 ng/ml, about 1000 ng/ml, about 1125 ng/ml or about 1250 ng/ml. In certain embodiments, the (Cavg) is obtained/attained within 3 hours after administration, preferably 2 hours, more preferably 1 hour after administering. In a preferred form of these embodiments, the (Cavg) is maintained over about 24 hours, and preferably over 12 weeks.

Methods of this invention may also involve administration of another component comprising an additional agent selected from an immunomodulatory agent; an antiviral agent; an inhibitor of HCV protease (other than VX-950); an inhibitor of another target in the HCV life cycle (other than NS3/4A protease); an inhibitor of internal ribosome entry, a broad-spectrum viral inhibitor; or combinations thereof. The additional agent is also selected from an inhibitor of viral cellular entry.

Such anti-viral agents include, but are not limited to, immunomodulatory agents, such as α-, β-, and γ-interferons or thymosin, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

Other agents (e.g., non-immunomodulatory or immunomodulatory compounds) may be used in combination with a compound of this invention include, but are not limited to, those specified in WO 02/18369, which is incorporated herein by reference (see, e.g., page 273, lines 9-22 and page 274, line 4 to page 276, line 11 this disclosure being specifically incorporated herein by reference).

Still other agents include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosures from those publications is referred to by reference to the publication number, but it should be noted that the disclosure of the compounds in particular is specifically incorporated herein by reference. Examples of such publications include U.S. Patent Application Publication Nos.: US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

Still other agents include, but are not limited to, Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.); COPEGUS® (Hoffmann-La Roche, Nutley, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al., “Efficacy and Safety of Pegylated (40-kd) Interferon alpha-2a Compared with Interferon alpha-2a in Noncirrhotic Patients with Chronic Hepatitis C,” Hepatology, 33, 433-438 (2001); consensus interferon (INFERGEN®)(Kao, J. H., et al., “Efficacy of Consensus Interferon in the Treatment of Chronic Hepatitis,” J. Gastroenterol. Hepatol., 15, 1418-1423 (2000); lymphoblastoid or “natural” interferon; interferon tau (Clayette, P. et al., “IFN-tau, A New Interferon Type I with Antiretroviral activity” Pathol. Biol. (Paris) 47, 553-559 (1999); interleukin-2 (Davis, G. L. et al., “Future Options for the Management of Hepatitis C.” Seminars in Liver Disease, 19, 103-112 (1999); Interleukin-6 (Davis et al., “Future Options for the Management of Hepatitis C,” Seminars in Liver Disease, 19, 103-112 (1999); interleukin-12 (Davis, G. L. et al., “Future Options for the Management of Hepatitis C.” Seminars in Liver Disease, 19, 103-112 (1999); and compounds that enhance the development of type 1 helper T cell response (Davis et al., “Future Options for the Management of Hepatitis C,” Seminars in Liver Disease, 19, 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod,” J. Am. Acad. Dermatol., 43 S6-11 (2000). See also, WO 02/18369, particularly page 272, line 15 to page 273, line 8, this disclosure being specifically incorporated herein by reference.

As is recognized by skilled practitioners, VX-950 is preferably administered orally. Interferon is not typically administered orally, although orally administered forms are in development. Nevertheless, nothing herein limits the methods or combinations of this invention to any specific dosage forms or regime. Thus, each component of a combination according to this invention may be administered separately, together, or in any combination thereof. As recognized by skilled practitioners, dosages of interferon are typically measured in IU (e.g., about 4 million IU to about 12 million IU). Interferon may also be dosed by micrograms. For example, a standard dose of Peg-Intron is 1.0-1.5 μg/kg/wk and of Pegasys is 180 μg/wk.

Typical peg-IFN and RBV treatment regimens include 12 weeks, 24 weeks, 36 weeks and 48 weeks treatments. Various types of peg-IFN are commercially available, for example, in vials as a prepared, premeasured solution or as a lyophilized (freeze-dried) powder with a separate diluent (mixing fluid). Pegylated interferon alfa-2b (Peg-Intron®) and alfa-2a (Pegasys®) are typical examples. Various types of interferon, including various dosage forms and formulation types, that can be employed in the invention are commercially available (see, e.g., specific examples of interferon described above). For example, various types of interferon are commercially available in vials as a prepared, premeasured solution or as a lyophilized (freeze-dried) powder with a separate diluent (mixing fluid). Pegylated interferon alfa-2b (Peg-Intron®) and alfa-2a (Pegasys®) vary from the other interferons by having molecules of polyethylene glycol (PEG) attached to them. The PEG is believed to cause the interferon to remain in the body longer and thus prolongs the effects of the interferon as well as its effectiveness. Pegylated interferon is generally administered by injection under the skin (subcutaneous). Pegasys® comes as an injectable solution in pre-filled syringes or in vials. The usual dose of Pegasys® is 180 μg, taken once a week. PEG-Intron® generally comes in a pre-filled pen that contains powder and sterile water; pushing down on the pen mixes them together. The dose of PEG-Intron® generally depends on weight-1.5 μg per kilogram (a range of between about 50 and about 150 μg total), taken once a week. In some embodiments, the dose of peg-interferon-alpha-2a is 180 mg/1.73 m², taken subcutaneously once a week. In certain embodiments, a pegylated interferon, e.g., pegylated interferon-alpha 2a or pegylated interfero-alpha 2b, is employed in the invention. Typically, interferon can be dosed according to the dosage regimens described in its commercial product labels.

Ribavirin is typically administered orally, and tablet forms of ribavirin are currently commercially available. General standard, daily dose of ribavirin tablets (e.g., about 200 mg tablets administered twice a day) is about 800 mg to about 1200 mg (according to the dosage regimens described in its commercial product labels). in some embodiments, the dose of ribavirin will be 15 mg/kg/day divided twice daily (capsule or solution) with a maximum of 1,200 mg if weight is 75 kg or 1,000 mg if <75 kg.

The methods herein may involve administration or co-administration to a patient a) combinations of VX-950 and another agent; or b) VX-950 in more than one dosage form. Co-administration includes administering each inhibitor in the same dosage form or in different dosage forms. When administered in different dosage forms, the inhibitors may be administered at different times, including about simultaneously or in any time period around administration of the other dosage forms. Separate dosage forms may be administered in any order. That is, any dosage forms may be administered prior to, together with, or following the other dosage forms.

In some aspects, the method includes the administration of agents to a patient over two phases, an initial phase and a secondary phase. For instance the initial phase can be a period of less than about 12 or 24 weeks and the secondary phase can be greater or equal to about 12 weeks, e.g., the secondary phase can be between about 12-36 weeks. In certain embodiments, the initial phase is 12 weeks. In certain embodiments, the initial phase is 24 weeks. In certain embodiments, the secondary phase is 12 weeks. In certain embodiments, the secondary phase is 24 weeks. In still other embodiments, the secondary phase is 36 weeks. In certain embodiments, the sum of the initial and secondary phase is about 24 to 48 weeks (such as 24, 36, or 48 weeks). In some embodiments, the initial and secondary phases can be identical in duration.

VX-950 may be administered in either the initial, secondary, or both phases. In some embodiments, VX-950 is administered only in the initial phase. When VX-950 is administered only in the initial phase, VX-950 may be administered alone or in combination with other agents and one or more agents are administered in the secondary phase. The other agents can be one or more anti-viral agents, one or more other agents described herein, or combinations thereof. In some embodiments, the specific agents administered in the initial and secondary phases are identical.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack comprising at least VX-950 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more of additional agent as described herein. The additional agent or agents may be provided in the same pack or in separate packs.

Another aspect of this involves a packaged kit for a patient to use in the treatment of HCV infection or in the prevention of HCV infection (or for use in another method of this invention), comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection.

Accordingly, this invention provides kits for the simultaneous or sequential administration of a dose of VX-950 (and optionally an additional agent). Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration.

In some embodiments, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

### Example 1

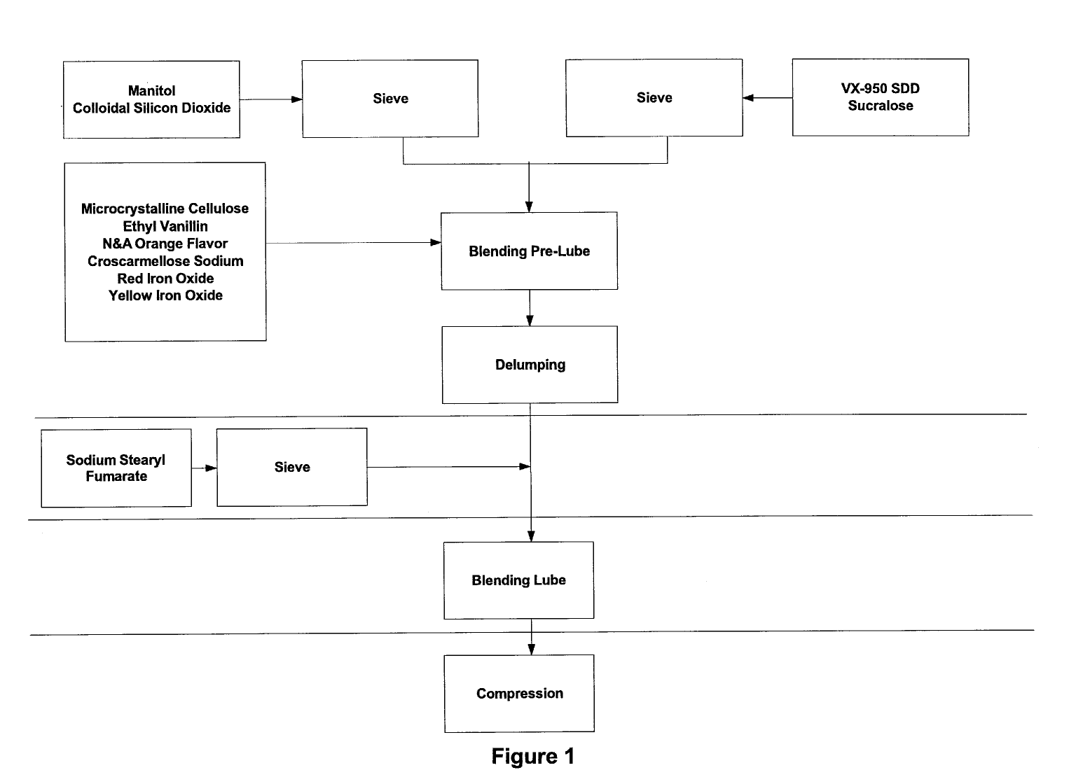

The manufacturing of VX-950 orange chewable tablet product is shown in FIG. 1.

The spray-dried dispersion of VX-950 is manufactured, which renders the drug substance amorphous to increase solubility and bioavailability. VX-950 drug substance, HPMCAS, and SLS are dissolved in solvent mixture containing methylene chloride/acetone/water. The solution is spray-dried to render the drug substance amorphous. The spray-dried intermediate is dried using a vacuum dryer to remove residual solvents. An example of the process of spray-dried dispersion can be found in International Publication Nos. WO 05/123076 and WO 07/109,605, which are incorporated herein by reference.

Blending of VX-950 spray-dried dispersion and selected compatible excipients is performed to form a tablet blend. VX-950 spray-dried dispersion and sucralose are co-screened through a 30 mesh screen; mannitol and colloidal silica are also co-screened through a 30 mesh screen. These binary mixtures are blended with remaining excipients of the formulation except the lubricant (sodium stearyl fumarate) in a stainless steel V-blender. De-lumping of the blend is performed using a co-mill at 5000 rpm through a 24R screen. Sodium stearyl fumarate is individually screened through a 60 mesh screen. (It would be recognized by one of ordinary skill in the art that alternate mesh sizes may be used. For example, the mesh may be a 50 mesh screen, a 60 mesh screen or a 70 mesh screen.) The lubricant is added to the blend, and additional blending takes place prior to final compression.

The tablet blend undergoes direct compression into 1000 mg round, orange, chewable VX-950 tablets (250 mg potency) or 400 mg round chewable VX-950 tablets (100 mg potency).

The composition of the chewable tablet is given below in table 1.

TABLE 1
Composition of VX-950 250 mg pediatric chewable tablet

| | Amount/tablet | | |
|---|---|---|---|
| Component | (mg) | | % w/w |
| VX-950 Spray Dried | 505.0 | | 50.50 |
| Dispersion (SDD)¹ | | | |
| Mannitol | 189.2 | | 18.92 |
| Micro-Crystalline Cellulose | 189.2 | | 18.92 |
| Sucralose | 20.0 | | 2.00 |
| Ethyl Vanillin | 5.20 | | 0.52 |
| N & A Orange Flavor | 21.4 | | 2.14 |
| Croscarmellose Sodium | 30.0 | | 3.00 |
| Colloidal Silica | 5.00 | | 0.50 |
| Red Iron Oxide | 1.50 | | 0.15 |
| Yellow Iron Oxide | 3.50 | | 0.35 |
| Sodium Stearyl Fumarate | 30.0 | | 3.00 |
| TOTAL | 1000 | mg | 100.00 |

¹Contains 250 mg of VX-950 assuming 100% purity in spray-dried dispersion

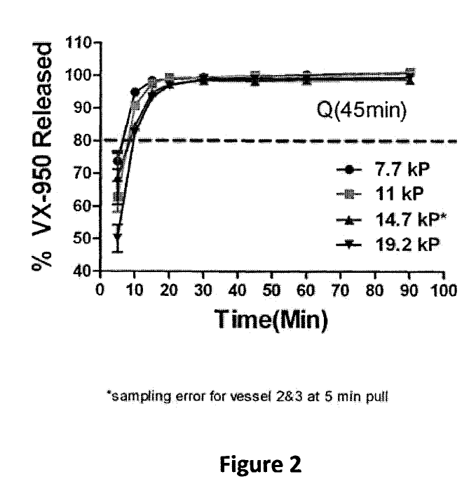

In tests performed to determine the effect of hardness on dissolution, a wide range of percent VX-950 is released during the first five minutes, demonstrating that an increase in hardness results in a slower initial release rate (FIG. 2). However, by 20 minutes, there are minimal differences among the tablet dissolution profiles with 14.7 kP and 19.2 kP exhibiting slightly slower dissolution profiles.

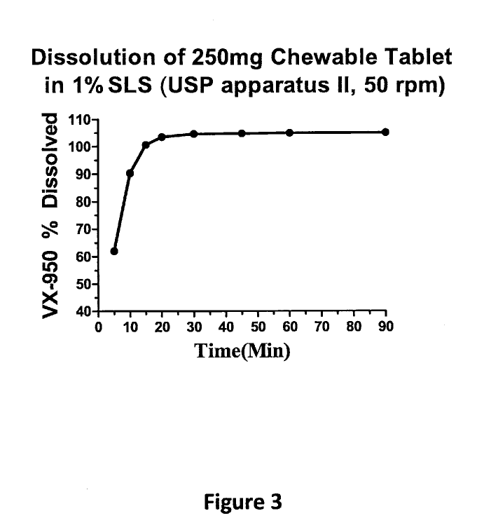

The dissolution method utilizes a 1% SLS dissolution medium, 900 mL volume and a paddle speed of 50 RPM. For the 250 mg chewable tablet, the dissolution profile is shown in FIG. 3.

### Example 2

Critical sensory attributes of a formulation comprising VX-950 were identified using trained sensory experts on the following negative sensory attributes were identified: bitterness (intense and lasting aftertaste); mouth irritation; and chalky/drying mouth feel (immediate and lasting feeling that makes the formulation (e.g. tablets) difficult to swallow).

Experienced pharmaceutical sensory panelists were administered three forms of formulation:

(a) the VX-950 tablet of Table 2 (“Adult Tablet”);

(b) a VX-950 formulation blended with sucralose & ethyl vanillin in Table 3; and

(c) a taste improving VX-950 formulation of Table 4.

The panelists were assigned unique code numbers; these numbers were used to track the individual panelist's results and their drug exposures.

TABLE 2
Composition of VX-950 250 mg Tablet

| | w/w % |
|---|---|
| Component | |
| VX-950 SDD | 75.64 |
| Lactose | 5.40 |
| Croscarmellose Sodium | 3.46 |
| Sodium Stearyl Fumerate | 0.23 |
| Melt granulate (surfactant, | 11.76 |
| binder, flow agent) | |
| Sodium Stearyl Fumerate | 3.51 |
| Total | 100 |
| Composition of the Melt | |
| Granulate | |
| Vitamin E TPGF | 29.38 |
| Microcrystalline cellulose | 41.25 |
| Croscarmellose Sodium | 19.58 |
| Colloidal Silicon Dioxide | 9.79 |
| Total | 100 |

TABLE 3

| Component | (wt. %) |
|---|---|
| VX-950 SDD¹ | 50.5 |
| Mannitol | 40.22 |
| Sucralose, micronized | 2.00 |
| Ethyl Vanillin | 0.40 |
| Croscarmellose Sodium (Ac- | 3.10 |
| Di-Sol) | |
| Silicon Dioxide | 0.78 |
| (Cab-O-Sil) | |
| Sodium Stearyl Fumerate | 3.00 |
| Total | 100.00 |

¹VX-950 SDD indicates for the compound of Formula I in a spray dried form.

TABLE 4

| Component | (wt. %) |
|---|---|
| VX-950 Placebo | 50.50 |
| Mannitol | 37.96 |
| Sucralose, micronized | 2.00 |
| Ethyl Vanillin | 0.520 |
| Croscarmellose Sodium (Ac- | 3.10 |
| Di-Sol) | |
| Silicon Dioxide | 0.78 |
| (Cab-O-Sil) | |
| Sodium Stearyl Fumerate | 3.00 |
| Natural & Artificial Orange | 2.14 |
| Flavor | |
| Total | 100.00 |

### Flavor Profile Definitions

Amplitude: Initial overall perception of balance and fullness, and is measured during the first 10-20 seconds of the evaluation and is an overall measure of the quality of the initial flavor.

### Amplitude Scale: 0=None

1=Low
2=Moderate
3=High
   Character Notes: Aromatics, basic tastes, and feeling factors (listed in order of appearance along with a measurement of strength).

### Intensity Scale: 0=None

1=Slight
2=Moderate
3=Strong
   The characteristics of a product that are evaluated as part of The Flavor Profile include a rating of the degree of blend and the amount of fullness present in the aroma and flavor as a whole. The integrative impression of blend and fullness is termed amplitude. When rated by a person trained in the method, amplitude measures the degree of integration of the perceptual experience, the complexity and structure of the aroma or flavor and is an overall measure of flavor quality.

Because people can perceive sensory effects at a slight intensity or above (≧1), the bitterness of VX-950 will be readily apparent to patients and is likely to be unacceptable, particularly to children.

For oral pharmaceuticals, both the initial flavor quality and the aftertaste flavor quality are important to patient acceptability, and therefore, it is important that each be evaluated.

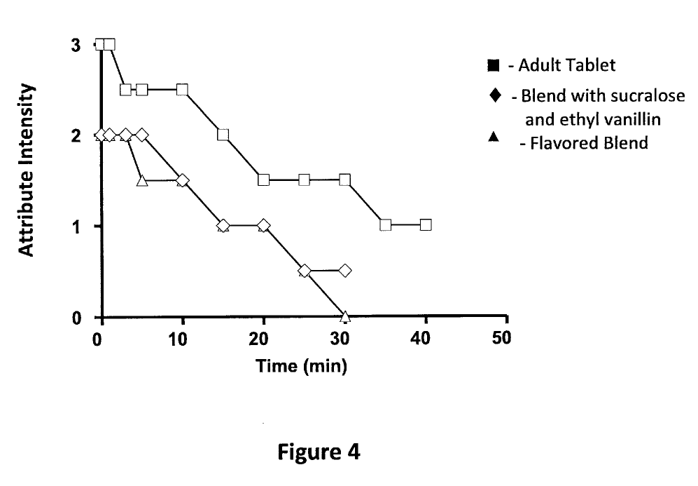

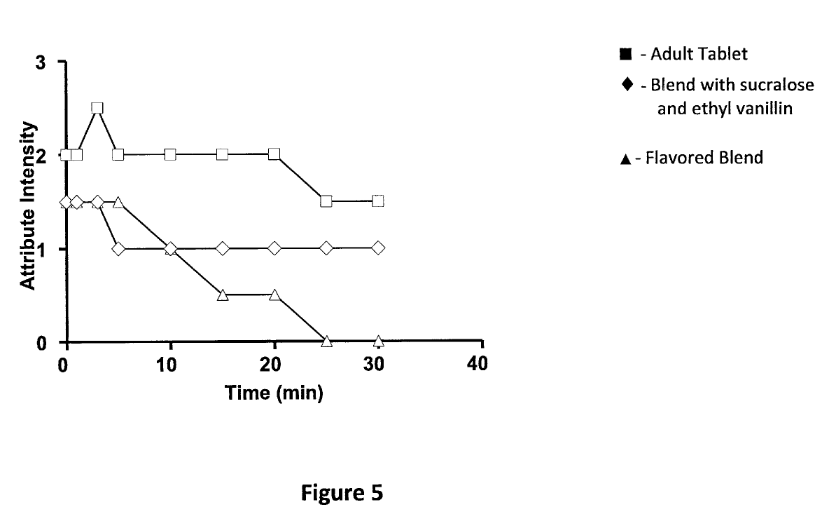

FIGS. 4 and 5 show the Flavor Profile results of the three different formulations above. Attribute intensity of 1 is considered the perception threshold.

### Example 3

TABLE 5
Formulation composition (250 mg strength in 1000 mg total tablet weight)

| Ingredient | Function | % wt. in Blend |
|---|---|---|
| VX-950 SDD | API | 50.5 |
| Mannitol | Filler | 17.59 |
| (Pearlitol SD100) | | |
| MCC | Filler | 20 |
| (Avicel PH 113) | | |
| Sucralose | Sweetener | 2 |
| N & A Orange | Flavor | 2.14 |
| flavor | | |
| Ethyl Vanillin | Flavor | 0.52 |
| Red Iron oxide | Colorant | 0.15 |
| Yellow Iron oxide | Colorant | 0.35 |
| Crosscarmellose | Disintegrant | 3 |
| sodium (Acdisol) | | |
| Sodium stearyl | Lubricant | 3 |
| fumarate | | |
| Colloidal silica | Glidant | 0.5 |
| (Cabosil) | | |

TABLE 6
Formulation composition (100 mg strength in 400 mg total tablet weight)

| Ingredient | Function |
|---|---|
| VX-950 SDD | API |
| Mannitol | Filler |
| (Pearlitol SD100) | |
| MCC | Filler |
| (Avicel PH 113) | |
| Sucralose | Sweetener |
| N & A Orange | Flavor |
| flavor | |
| Ethyl Vanillin | Flavor |
| Red Iron oxide | Colorant |
| Yellow Iron oxide | Colorant |
| Crosscarmellose | Disintegrant |
| sodium (Acdisol) | |
| Sodium stearyl | Lubricant |
| fumarate | |
| Colloidal silica | Glidant |
| (Cabosil) | |

### Example 4

A tablet of the present invention and the 250-mg VX-950 SDD powder, whose composition is shown in Table 2 were evaluated in a similar procedure as Example 2 in different sensory attributes, including chalky/dry mouth feel and bitterness. The results are shown in Tables 7 and 8.

TABLE 7
Flavor Profile of VX-950 SDD powder (250 mg)

| | | 1 | 3 | 5 | 10 | 15 | 20 | 25 | 30 |
|---|---|---|---|---|---|---|---|---|---|
| | Initial | Min | Min | Min | Min | Min | Min | Min | Min |
| Bitter | 3 | 3 | 2.5 | 2 | 2 | 1.5 | 1.5 | 1 | 1 |
| Chalk/Dry | 1.5 | 1.5 | 1.5 | 1.5 | 1.5 | 1.5 | 1 | 1 | 0.5 |
| Mouth | | | | | | | | | |

TABLE 8
Flavor profile of VX-950 Palatable Tablet (250 mg)

| | | 1 | 3 | 5 | 10 | 15 | 20 | 25 | 30 |
|---|---|---|---|---|---|---|---|---|---|
| | Initial | Min | Min | Min | Min | Min | Min | Min | Min |
| Bitter | 2 | 2 | 2 | 1.5 | 1.5 | 1 | 1 | 0.5 | 0.5 |
| Chalk/Dry | 1.5 | 1 | 1.5 | 1.5 | 1 | 0.5-1 | 0.5 | 0.5 | — |
| Mouth | | | | | | | | | |

What is claimed is:

1. A pharmaceutical formulation comprising:
VX-950; and
a taste improving composition.

2. The pharmaceutical formulation of claim 1, wherein the wt. % ratio of VX-950 with respect to the taste improving composition ranges from about 20:1 to about 1:2.

3. The pharmaceutical formulation of claim 2, wherein the wt. % ratio of VX-950 with respect to the taste improving composition ranges from about 15:1 to about 10:1.

4. The pharmaceutical formulation of claim 1, wherein VX-950 is in an amorphous form.

5. The pharmaceutical formulation of claim 4, wherein VX-950 is spray-dried with a polymer.

6. The pharmaceutical formulation of claim 1, wherein the taste improving composition comprises a flavoring agent and a sweetener.

7. The pharmaceutical formulation of claim 6, wherein the flavoring agent is a natural flavor, an artificial flavor, or both.

8. The pharmaceutical formulation of claim 7, wherein the flavoring agent includes one or more ingredients selected from the group consisting of: spearmint oil, cinnamon oil, oil of wintergreen, peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, and natural and artificial orange flavor.

9. The pharmaceutical formulation of claim 8, wherein the flavor agent is ethyl vanillin, a fruit flavor or both.

10. The pharmaceutical formulation of claim 9, wherein the fruit flavor includes one or more ingredients selected from the group consisting of natural and/or artificial flavor of apple, pear, peach, orange, grape, strawberry, raspberry, cherry, plum, pineapple, and apricot.

11. The pharmaceutical formulation of claim 10, wherein the fruit flavor is natural and artificial orange.

12. The pharmaceutical formulation of claim 6, comprising from about 0 wt. % to about 5 wt. % of the flavor agent.

13. The pharmaceutical formulation of claim 12, comprising from about 1 wt. % to about 2.5 wt. % of the flavoring agent.

14. The pharmaceutical formulation of claim 6, wherein the sweetener is selected from one or more from the group consisting of the following: glucose, sucrose, maltose, mannose, dextrose, fructose, lactose, trehalose, maltitol, lactitol, xylitol, sorbitol, mannitol, tagatose, glycerin, erythritol, isomalt, sucralose, aspartane, neotame, alitame, neohesperidin dihydrochalcone, sodium cyclamate, thaumatin, acesulfame potassium, saccharin and saccharin sodium.

15. The pharmaceutical formulation of claim 14, wherein the sweetener is sucralose, aspartame or both.

16. The pharmaceutical formulation of claim 14, wherein the sweetener is sucralose.

17. The pharmaceutical formulation of claim 6, comprising from about 1 wt. % to about 4 wt. % of the sweetener.

18. The pharmaceutical formulation of claim 17 comprising from about 1 wt. % to about 2 wt. % of the sweetener.

19. The pharmaceutical formulation of claim 1, comprising from about 20 wt. % to about 80 wt. % of VX-950.

20. The pharmaceutical formulation of claim 19 comprising from about 40 wt. % to about 60 wt. % of VX-950.

21. The pharmaceutical formulation of claim 19 comprising from about 50 wt. % of VX-950.

22. The pharmaceutical formulation of claim 1, further comprising one or more excipients.

23. The pharmaceutical formulation of claim 22, wherein the one or more excipients is selected from the group consisting of: a filler, a glidant, a lubricant, a disintegrant and a colorant.

24. The pharmaceutical formulation of claim 23, wherein the disintergrant comprises one or more ingredients selected from the group consisting of: croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate.

25. The pharmaceutical formulation of claim 24, wherein the disintegrant is croscarmellose sodium.

26. The pharmaceutical formulation of claim 24 or 25 comprising from about 1 wt. % to about 5 wt. % of the disintegrant.

27. The pharmaceutical formulation of claim 24 or 25 comprising about 3 wt. % of the disintegrant.

28. The pharmaceutical formulation of claim 23, wherein the lubricant comprising one or more ingredients selected from the group consisting of: talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, and sodium benzoate.

29. The pharmaceutical formulation of claim 28, wherein the lubricant is sodium stearyl fumarate.

30. The pharmaceutical formulation of claim 28 comprising from about 1 wt. % to about 5 wt. % of the lubricant.

31. The pharmaceutical formulation of claim 28 comprising about 3 wt. % of the lubricant.

32. The pharmaceutical formulation of claim 23, wherein the filler includes one or more ingredients selected from the group consisting of: lactose, dextrose, maltodextrin, sorbitol, xylitol, mannitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate.

33. The pharmaceutical formulation of claim 32, wherein the filler is mannitol, microcrystalline cellulose or both.

34. The pharmaceutical formulation of claim 32, wherein the filler comprises from about 20 wt. % to about 50 wt. %.

35. The pharmaceutical formulation of claim 32, wherein the filler comprises from about 35 wt. % to about 45 wt. %.

36. The pharmaceutical formulation of claim 32, wherein the filler comprises from about 38 wt. % to about 41 wt. %.

37. The pharmaceutical formulation of claim 23, wherein the one or more excipients further comprises a colorant.

38. The pharmaceutical formulation of claim 37, wherein the colorant includes one or more ingredients selected from the group consisting of red, black and yellow iron oxides, and FD & C dyes.

39. The pharmaceutical formulation of claim 23, wherein the one or more excipients is selected from the group consisting of: croscarmellose sodium, sodium stearyl fumarate, microcrystalline cellulose, red and yellow iron oxides, mannitol, colloidal silica and a combination thereof.

40. The pharmaceutical formulation of claim 1, wherein the pharmaceutical formulation is in a form of a capsule, tablet, pill, powder, granule, or aqueous suspension or solution.

41. The pharmaceutical formulation of claim 40, wherein the formulation is in a form of a tablet.

42. The pharmaceutical formulation of claim 41, wherein the tablet is chewable.

43. The pharmaceutical formulation of claim 1, wherein the intensity of the bitterness of the pharmaceutical formulation, when administered, is at least 30% less than a VX-950 formulation without the taste improving composition.

44. The pharmaceutical formulation of claim 1, wherein the intensity of the bitterness of the pharmaceutical formulation is at least 50%, 10 min after administered, less than a VX-950 formulation without the taste improving composition.

45. The pharmaceutical formulation of claim 1, wherein the intensity of the chalky/dry mouthfeel of the pharmaceutical formulation is at least 50% less than a VX-950 formulation without the taste improving composition, 10 min after administration.

46. A method of preparing a pharmaceutical formulation comprising:
a) blending VX-950 with a taste improving composition and one or more excipients;
b) forming a blended mixture; and
c) lubricating the blended mixture.

47. The method of claim 46, wherein blending an API with a palatable composition and forming a blended mixture includes delumping VX-950 and the taste improving composition.

48. The method of claim 46, wherein the intensity of the bitterness of the pharmaceutical formulation, when administered, is at least 30% less than a VX-950 formulation without the taste improving composition.

49. The method of claim 46, wherein the intensity of the chalky/dry mouthfeel of the pharmaceutical formulation is at least 50% less than a VX-950 formulation without the taste improving composition, 10 min after administration.

50. The method of claim 46, wherein the intensity of the chalky/dry mouthfeel of the pharmaceutical formulation is at least 50% less than a VX-950 formulation without the taste improving composition, 10 min after administration.

51. A pharmaceutical formulation comprising:
a) a means for blending VX-950 with a taste improving composition and one or more excipients;
b) a means for forming a blended mixture; and
c) a means for lubricating the blended mixture.

52. The pharmaceutical formulation of claim 51, wherein the intensity of the bitterness of the pharmaceutical formulation, when administered, is at least 30% less than a VX-950 formulation without the taste improving composition.

53. The pharmaceutical formulation of claim 51, wherein the intensity of the chalky/dry mouthfeel of the pharmaceutical formulation is at least 50% less than a VX-950 formulation without the taste improving composition, 10 min after administration.

54. A pharmaceutical formulation comprising:
a) VX-950 in a spray-dried dispersion;
b) ethyl vanillin;
c) natural and artificial orange flavor; and
d) sucralose.

55. A method of administering the pharmaceutical formulation of claim 1 to a patient infected with hepatitis C.

56. The method of claim 55, further comprising administering one or more additional antiviral agents.

57. The method of claim 56, wherein the one or more antiviral agents are pegylated-interferon and ribavirin.

58. The method of claim 55, wherein the pharmaceutical formulation per administration is in an amount of about 250 mg to about 2250 mg VX-950.

59. The method of claim 55, wherein the pharmaceutical formulation is administered:
a) in an amount of 250 mg VX-950;
b) in an amount of 300 mg VX-950;
c) in an amount of 400 mg VX-950;
d) in an amount of 450 mg VX-950;
e) in an amount of 500 mg VX-950;
f) in an amount of 600 mg VX-950;
g) in an amount of 650 mg VX-950;
h) in an amount of 750 mg VX-950;
i) in an amount of 850 mg VX-950;
j) in an amount of 1000 mg VX-950;
k) in an amount of 1250 mg VX-950
l) in an amount of 2250 mg VX-950.

60. The method of claim 55, wherein the pharmaceutical formulation per administration is in an amount of about 10 mg to about 20 mg VX-950 per kilogram of body weight.

61. The method of claim 55, wherein the pharmaceutical formulation is administered:
a) in an amount of about 15 mg per kilogram of body weight;
b) in an amount of about 16 mg per kilogram of body weight;
c) in an amount of about 18 mg per kilogram of body weight.

62. The method of claim 55, wherein the pharmaceutical formulation is administered once per day, twice per day or three times per day.
